(19) United States
(12) Patent Application Publication
(10) Pub. No.: US 2009/0198258 A1
(43) Pub. Date: Aug. 6, 2009

(54) Curved arthroscopic guide

(76) Inventor: William Buchanan Workman, Lafayette, CA (US)

Correspondence Address: PATENT LAW OFFICE OF DAVID G. BECK, P. O. BOX 1146, MILL VALLEY, CA, 94942, US

(21) Appl. No.: 12/012,625

(22) Filed: Feb. 1, 2008

(51) Int. Cl. A61B 17/04

(52) U.S. Cl. 606/148

(57) ABSTRACT

A rigid, curved and hollow guide (100) that can be used to facilitate a variety of arthroscopic surgeries is provided. A longitudinal bore (201) extends from end to end within the guide, at least a portion of which is curved. At least one slotted region (303) extends along at least a portion of the longitudinal bore.

### CROSS-REFERENCES TO RELATED APPLICATIONS

This application is a continuation-in-part of U.S. patent application Ser. No. ______, filed Jan. 30, 2008 and entitled Curved Suture Anchor Guide and Method of Use (Attorney Docket Number WW001), the disclosure of which is incorporated herein by reference for any and all purposes.

### FIELD OF THE INVENTION

The present invention relates generally to orthopedic surgery and, more particularly, to an improved arthroscopic surgical instrument.

### BACKGROUND OF THE INVENTION

Arthroscopy is a diagnostic and surgical technique that has been in use for decades. In its simplest form, it provides a minimally invasive technique for diagnosing any of a variety of disorders, primarily disorders involving a joint such as the knee, shoulder, elbow, hip, ankle and wrist. During diagnosis, one or more small incisions are made around the joint or other area in question and a small, flexible fiberoptic camera, also referred to as an arthroscope, is inserted into the region thereby allowing the physician to inspect and evaluate the injured region while causing minimal damage to surrounding tissues. Once the problem has been assessed, the physician can determine the best course of action, whether that is surgery or a non-surgical approach such as physical and/or drug therapy. If surgery is required, in a large percentage of cases involving the musculoskeletal system the orthopedic surgeon has a choice between using conventional ‘open’ surgical techniques or arthroscopic techniques. The latter approach uses specially designed surgical instruments that can be inserted into small incisions surrounding the region of interest and used to remove and/or repair damaged tissue. As this approach causes minimal damage to surrounding tissue, recovery time is accelerated, post surgery motion is often better than that achievable through open surgery, and scarring is reduced.

Although arthroscopic surgery offers a number of benefits to the patient, arthroscopic surgical techniques are very demanding since the surgeon must operate remotely, i.e., via an arthroscopic camera. Additionally, in order to maximize the benefits offered by this approach, the surgeon is continually trying to minimize collateral tissue damage by reducing the number and size of incisions through which the operation is performed. Clearly these goals are most difficult to achieve for surgeons with less experience, either because they are relatively new to the practice of medicine or because they are infrequently required to perform a particular arthroscopic technique. Accordingly, arthroscopic surgical instruments that can simplify arthroscopic surgery are in great demand. The present invention provides such a surgical instrument.

### SUMMARY OF THE INVENTION

The present invention provides a rigid, curved and hollow guide that can be used to facilitate a variety of arthroscopic surgeries. Due to at least a section of the guide being curved, various regions within the surgical site can be easily accessed by simply rotating and/or repositioning the guide. As a result of this capability, fewer and smaller incisions are required during the operation, thus leading to less collateral tissue damage, quicker recovery time and fewer associated cosmetic issues.

In one embodiment of the invention, a curved arthroscopic guide is provided, the instrument comprised of a curved longitudinal bore that extends from end to end, and at least one adjoining slotted region that extends along at least a portion of the curved longitudinal bore. In one aspect, the curved longitudinal bore is suitably sized to pass a suture anchor and with a sufficiently large radius of curvature to allow passage of the suture anchor. The curved arthroscopic guide can further be comprised of an angled tip at the distal end of the guide, and a handle disposed on the proximal end of the guide. In another aspect, the radius of curvature corresponding to the curved longitudinal bore is sufficiently large to allow passage of the arthroscopic guide through a portal.

In another embodiment of the invention, a curved arthroscopic guide is provided, the instrument comprised of a longitudinal bore that extends from end to end and is comprised of a first straight portion and a second curved portion, and at least one adjoining slotted region that extends along at least a portion of the longitudinal bore. In one aspect, the longitudinal bore is suitably sized to pass a suture anchor and the curved portion of the longitudinal bore has a sufficiently large radius of curvature to allow passage of the suture anchor. The curved arthroscopic guide can further be comprised of an angled tip at the distal end of the guide, and a handle disposed on the proximal end of the guide. In another aspect, the radius of curvature corresponding to the curved portion of the longitudinal bore is sufficiently large to allow passage of the arthroscopic guide through a portal.

In another embodiment of the invention, a curved arthroscopic guide is provided, the instrument comprised of a longitudinal bore that extends from end to end and is suitably sized to pass a suture anchor, wherein at least a portion of the longitudinal bore is curved with a radius of curvature sufficiently large to allow passage of the suture anchor, the guide further comprising first and second adjoining slotted regions that extend along the longitudinal bore.

In another embodiment of the invention, an arthroscopic kit is provided, the kit comprised of a curved arthroscopic guide and an arthroscopic tool, wherein the curved arthroscopic guide is comprised of a longitudinal bore that extends from end to end and of which at least a portion is curved, and at least one adjoining slotted region extending along the longitudinal bore, and wherein the arthroscopic tool is comprised of a flexible tool shaft with at least one rail, the tool shaft and the at least one rail configured to fit within the longitudinal bore and the at least one slotted region, respectively.

A further understanding of the nature and advantages of the present invention may be realized by reference to the remaining portions of the specification and the drawings.

### BRIEF DESCRIPTION OF THE DRAWINGS

FIG. 1 is a side view of a curved guide in accordance with a preferred embodiment of the invention;

FIG. 2 is a cut-away view of the curved guide shown in FIG. 1;

FIG. 3 is a cross-sectional view of the guide shaft of the embodiment shown in FIG. 1;

FIG. 4 is a cut-away, top-down view of the curved guide shown in FIG. 1;

FIG. 5 is an illustration of a flexible tool for use with the curved guide of the invention;

FIG. 6 is an end view of the flexible tool shown in FIG. 5;

FIG. 7 is a side view of an exemplary tool tip for use with the flexible tool shown in FIG. 5;

FIG. 8 is a cut-away view of a curved guide in accordance with an alternate embodiment of the invention;

FIG. 9 is a cross-sectional view of the guide shaft of the embodiment shown in FIG. 8;

FIG. 10 is a cross-sectional view of a flexible tool with a single rail;

FIG. 11 is a cross-sectional view of a guide shaft with a single slot; and

FIG. 12 is a cross-sectional view of a guide shaft with four slots.

### DESCRIPTION OF THE SPECIFIC EMBODIMENTS

In traditional arthroscopic surgery, the surgeon uses a variety of tools passed through one or more cannulas to remove and/or repair damaged tissue. Typically these tools are rigid and designed to be passed singularly through a cannula, the surgeon manipulating the tool using the tool's handle. In an alternate approach described in detail in co-pending U.S. patent application Ser. No. ______, filed Jan. 30, 2008 and entitled Curved Suture Anchor Guide and Method of Use, a technique for using tools in combination with a curved guide is disclosed, this alternative approach intended to provide access to regions that would not be accessible, or at angles that would not be achievable, with a non-curved guide. As a result of this approach, fewer and smaller incisions are required during an operation, thus leading to less collateral tissue damage, quicker recovery, and fewer cosmetic issues. A difficulty associated with this approach, however, is that in some instances the tool may rotate independently of the guide, thus making it hard for the surgeon to control the tool with the desired level of precision.

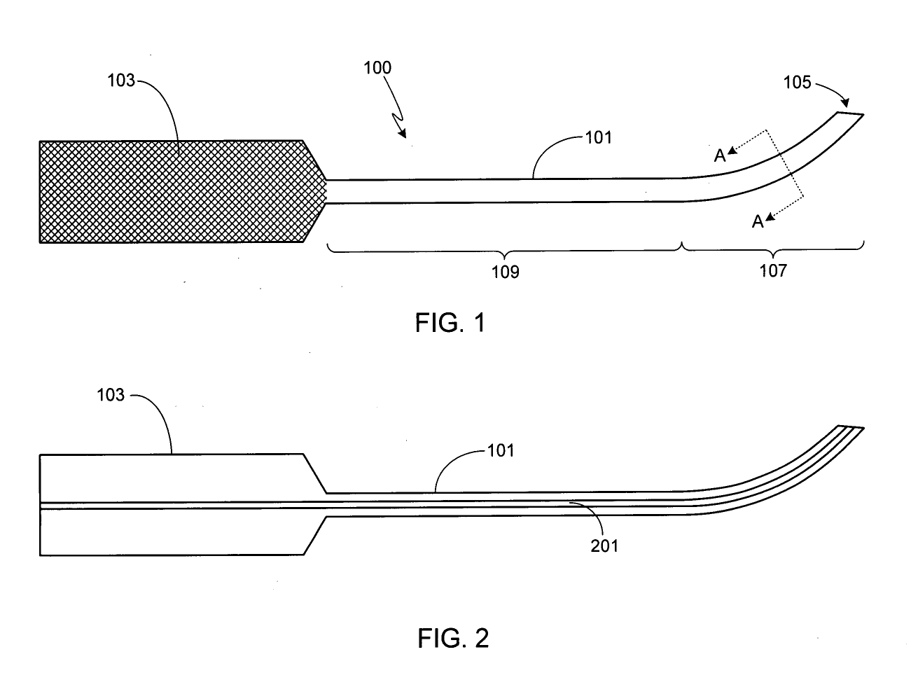

FIGS. 1-4 illustrate a rigid, curved and hollow guide 100 in accordance with a preferred embodiment of the invention. As shown in the side view of FIG. 1, hollow guide 100 is comprised of a hollow guide shaft 101 coupled to a handle 103. Preferably tip 105 of shaft 101 is angled, more preferably with a tip angle of approximately 45 degrees. In the cut-away view of FIG. 2, bore 201 is shown extending longitudinally along the entire length of guide shaft 101 and extending through handle 103, thus allowing sutures, suture anchors, and various tools to be passed through guide 100.

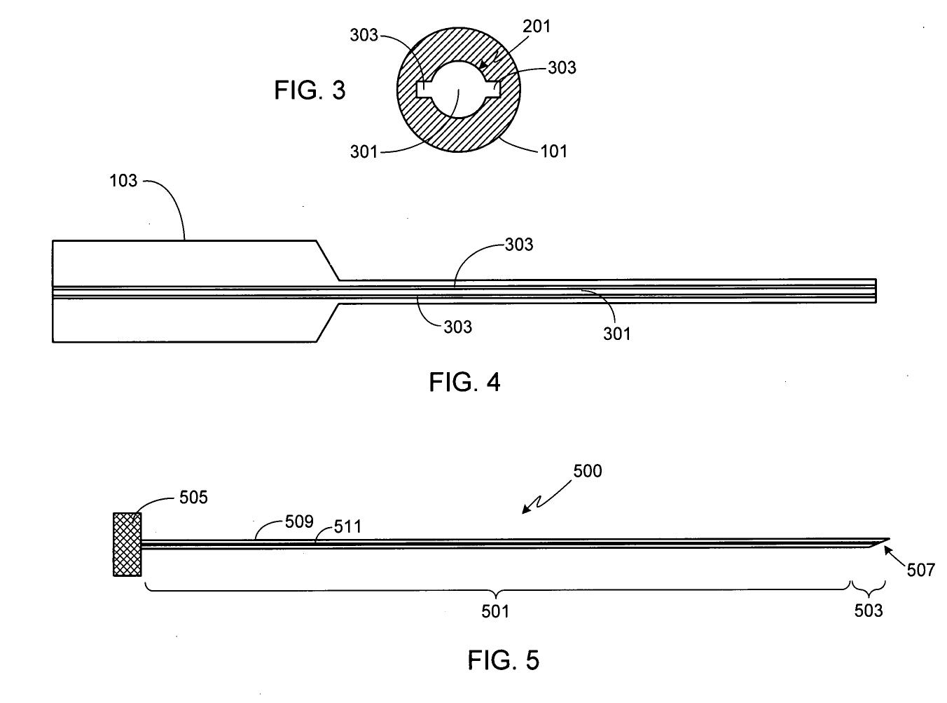

FIG. 3 is a cross-sectional view of guide shaft 101 taken at plane A-A. As shown, bore 201 is comprised of a central region 301, preferably cylindrical as shown, and a pair of slotted regions 303 that adjoin region 301. In this embodiment there are two slots 303, one on either side of cylindrical region 301. Preferably, and for the reasons described in detail below, slots 303 run the full length of bore 201 as shown in the cut-away view of FIG. 4, this figure taken from a top down perspective of guide 100 rather than a side perspective as used in FIG. 2.

As described in co-pending U.S. patent application Ser. No. ______, any of a variety of tools can be designed to pass through the curved guide. An exemplary tool is shown in FIG. 5, tool 500 including a flexible shaft portion 501 and preferably a rigid tip portion 503 for improved tool tip control. It will be appreciated that the rigid portion 503 of the tool must be short enough to slide through the curved guide and as such, either the radius of curvature of the curved guide must be selected based on the length of rigid portion 503, or the length of rigid portion 503 must be no longer than that for which the curved guide was designed. Preferably tool 500 also includes a tool handle 505. Although any of a variety of tips can be integrated into tool 500, in the exemplary embodiment shown in FIG. 5, tip 507 is angled, thus providing chisel-like functionality, with sharpened edges to aid in tissue liberation. Tip 507 can also include an abrading surface.

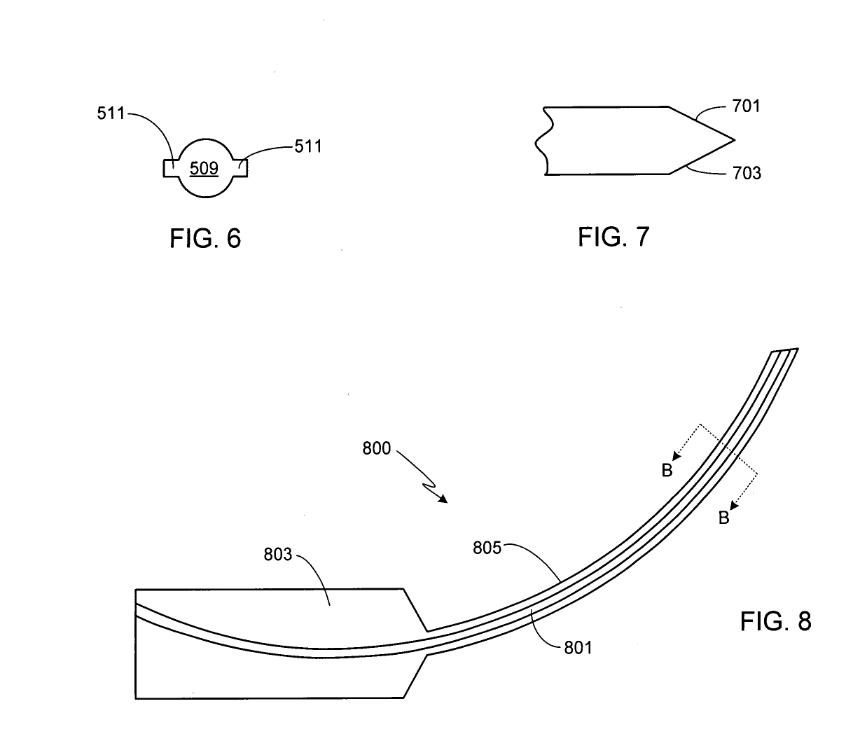

Along the length of shaft 509 of tool 500 run a pair of extended regions 511, referred to herein as rails. The end view of tool 500 shown in FIG. 6 more clearly illustrates rails 511. Rails 511 are designed and configured to slide through slots 303 when tool 500 is inserted through curved guide 100. Accordingly, even though tool 500 is flexible, during use it cannot rotate within curved guide 100 due to the interplay between rails 511 and complementary slots 303. As a result, the surgeon is provided with more precise control of tool 500. Note that if desired, tool 500 can be withdrawn from curved guide 100, rotated by 180 degrees, and reinserted within curved guide 100, thereby allowing the surgeon to use a different face and/or aspect of tool 500. For example and as illustrated in FIG. 7, if the tip of tool 500 has a pair of faces 701/703 of different abrasion levels, the surgeon is able to select and maintain the desired abrasive surface by selecting the orientation of tool 500 in curved guide 100.

In addition to maintaining a specific tool orientation within the guide, slots 303 also provide a benefit when passing a pre-threaded anchor through the guide. More specifically, when an anchor is inserted into the guide, followed by an anchor driver, the suture rests within slots 303, preferably with one leg of the suture within each slot. As a result, the risk of the pre-threaded suture anchor and/or the anchor driver becoming wedged within the guide during use is minimized.

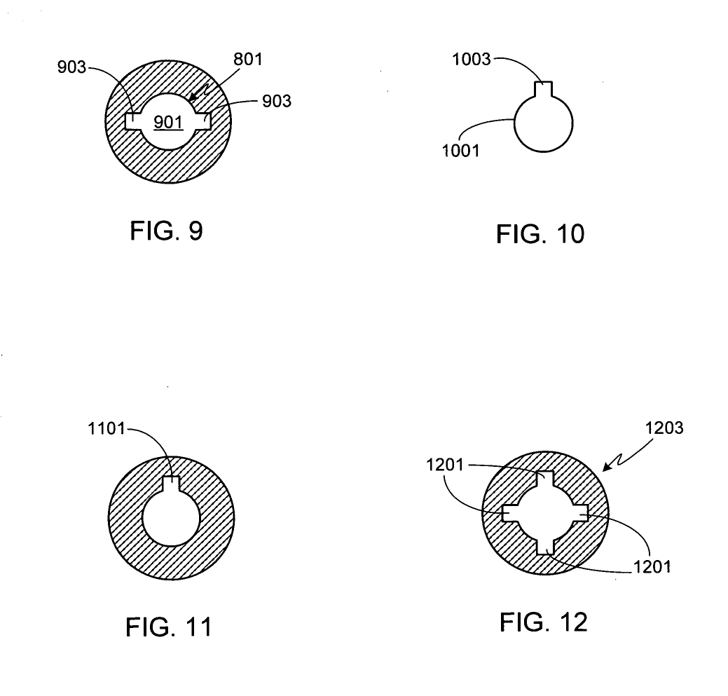

In the embodiment illustrated in FIGS. 1-4, only an end portion 107 of guide shaft 101 is curved, the remaining portion 109 being straight. It will be appreciated that a curved guide utilizing the invention can also be continuously curved, for example as shown in FIG. 8. As shown, the entire bore 801 of guide 800 is curved, including the portion passing through handle 803. As in the previous embodiment and as illustrated in the cross-sectional view of guide shaft 805 taken at plane B-B, i.e., FIG. 9, bore 801 is comprised of a central region 901, preferably cylindrical, and a pair of adjoining slotted regions 903.

It should be understood that although the preferred embodiments of the curved guide shown above include a pair of slots and that the tools described for use with these guides include a pair of rails designed to match the slots, the invention can be applied to other configurations that use different slot and rail combinations and still gain the benefits of the invention. For example, FIG. 10 is a cross-sectional view of a tool shaft 1001 with a single rail 1003. Rail 1003 can fit within either slot of the previously described curved guides, i.e., slots 303 or slots 903, and still function properly. Alternately, a tool with a single rail can be used with a curved guide that has a single slot, e.g., slot 1101 shown in the guide shaft cross-sectional view of FIG. 11. Note that in this case the tool will only fit within the guide in a single orientation, as opposed to two orientations as with the previous embodiments. Additionally, the rail and slot system can be designed to provide more than one or two configurations. For example, if the curved guide has four slots 1201 as shown in the cross-sectional view of guide shaft 1203 (FIG. 12) and the tool shaft has a single rail 1003 as shown in the cross-sectional view of tool shaft 1001 (FIG. 10), the tool can be inserted within the guide in four different orientations. It will be appreciated that there are countless variations of the rail and slot system of the invention that provide the desired tool stability for a flexible tool and a curved guide.

In addition to the above, in at least one embodiment the slots, the rails, or both do not run the entire length of the guide bore/tool shaft. For example, hollow guide 100 (or hollow guide 800) can be configured with one or more slots near the proximal end of the guide, typically within handle 103. Assuming the flexible tool only has a complementary rail or rails at the proximal end of the tool shaft, the flexible tool will be free to rotate within the guide until the tool has been inserted far enough into the guide to engage the rail(s) within the slot(s). The same result occurs if the guide has slots running the entire length of the hollow guide, but the flexible tool only has a complementary rail or rails at the proximal end of the tool shaft. A benefit of this embodiment is that it allows the surgeon to easily change the orientation of the tool relative to the curved guide since the tool does not have to be completely withdrawn from the guide to be rotated.

As will be understood by those familiar with the art, the present invention may be embodied in other specific forms without departing from the spirit or essential characteristics thereof. Accordingly, the disclosures and descriptions herein are intended to be illustrative, but not limiting, of the scope of the invention that is set forth in the following claims.

What is claimed is:

1. A curved arthroscopic guide comprised of a curved longitudinal bore, said curved longitudinal bore extending from a proximal end surface of said curved arthroscopic guide to a distal end surface of said curved arthroscopic guide, said curved arthroscopic guide further comprised of at least one slotted region extending along at least a portion of said curved longitudinal bore, wherein said at least one slotted region adjoins said portion of said curved longitudinal bore.

2. The curved arthroscopic guide of claim 1, wherein said curved longitudinal bore is suitably sized to pass a suture anchor, and wherein a radius of curvature corresponding to said curved longitudinal bore is sufficiently large to allow passage of said suture anchor.

3. The curved arthroscopic guide of claim 2, wherein said radius of curvature is greater than or equal to 12.5 centimeters.

4. The curved arthroscopic guide of claim 1, further comprising an angled tip at said distal end surface of said curved arthroscopic guide.

5. The curved arthroscopic guide of claim 1, further comprising a handle disposed on a proximal end portion of said curved arthroscopic guide, wherein said curved longitudinal bore passes through said handle.

6. The curved arthroscopic guide of claim 1, wherein said at least one slotted region extends along and adjoins to an entire length of said curved longitudinal bore.

7. The curved arthroscopic guide of claim 1, wherein said at least one slotted region is comprised of a first slotted region and a second slotted region.

8. The curved arthroscopic guide of claim 1, wherein a radius of curvature corresponding to said curved longitudinal bore is sufficiently large to allow passage of said curved arthroscopic guide through a portal.

9. A curved arthroscopic guide comprised of a longitudinal bore, said longitudinal bore extending from a proximal end surface of said curved arthroscopic guide to a distal end surface of said curved arthroscopic guide, wherein said longitudinal bore has a first portion proximate to said proximal end surface and a second portion proximate to said distal end surface, wherein said first portion of said longitudinal bore is straight and said second portion of said longitudinal bore is curved, and wherein said curved arthroscopic guide is further comprised of at least one slotted region extending along at least a portion of said longitudinal bore, wherein said at least one slotted region adjoins said portion of said longitudinal bore.

10. The curved arthroscopic guide of claim 9, wherein said longitudinal bore is suitably sized to pass a suture anchor, and wherein a radius of curvature corresponding to said second portion of said longitudinal bore is sufficiently large to allow passage of said suture anchor.

11. The curved arthroscopic guide of claim 10, wherein said radius of curvature is greater than or equal to 12.5 centimeters.

12. The curved arthroscopic guide of claim 9, further comprising an angled tip at said distal end surface of said curved arthroscopic guide.

13. The curved arthroscopic guide of claim 9, further comprising a handle disposed on a proximal end portion of said curved arthroscopic guide, wherein said longitudinal bore passes through said handle.

14. The curved arthroscopic guide of claim 9, wherein said at least one slotted region extends along and adjoins to an entire length of said longitudinal bore.

15. The curved arthroscopic guide of claim 9, wherein said at least one slotted region is comprised of a first slotted region and a second slotted region.

16. The curved arthroscopic guide of claim 9, wherein a radius of curvature corresponding to said second portion of said longitudinal bore is sufficiently large to allow passage of said curved arthroscopic guide through a portal.

17. A curved arthroscopic guide comprised of a longitudinal bore, wherein said longitudinal bore extends from a proximal end surface of said curved arthroscopic guide to a distal end surface of said curved arthroscopic guide, wherein said longitudinal bore is suitably sized to pass a suture anchor, wherein at least a portion of said longitudinal bore is curved with a radius of curvature sufficiently large to allow passage of said suture anchor, wherein said curved arthroscopic guide is further comprised of a first slotted region extending along said longitudinal bore and a second slotted region extending along said longitudinal bore, and wherein said first and second slotted regions adjoin said longitudinal bore.

18. An arthroscopic surgical kit comprising:
a curved arthroscopic guide comprised of a longitudinal bore, wherein said longitudinal bore extends from a proximal end surface of said curved arthroscopic guide to a distal end surface of said curved arthroscopic guide, wherein at least a portion of said longitudinal bore is curved, wherein said curved arthroscopic guide is further comprised of at least one slotted region extending along said longitudinal bore, wherein said at least one slotted region adjoins said longitudinal bore; and
an arthroscopic tool comprised of a flexible tool shaft configured to fit within said longitudinal bore, said arthroscopic tool further comprised of at least one rail extending along said flexible tool shaft and configured to fit within said at least one slotted region.

19. The arthroscopic surgical kit of claim 18, wherein said at least one slotted region is comprised of a first slotted region and a second slotted region.
